(19) United States
(12) Patent Application Publication
(10) Pub. No.: US 2005/0152845 A1
(43) Pub. Date: Jul. 14, 2005

(54) Amorphous fluticasone 2-furoate, pharmaceutical compositions thereof and its conversion to the crystalline unsolvated form

(76) Inventors: Keith Biggadike, Stevenage (GB); Steven John Coote, Stevenage (GB); Brian Noga, Research Triangle Park, NC (US); Michiel Mary Van Oort, Research Triangle Park, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/503,394

(22) PCT Filed: Feb. 4, 2003
(86) PCT No.: PCT/GB03/00461

Related U.S. Application Data
(60) Provisional application No. 60/354,143, filed on Feb. 4, 2002.

(51) Int. Cl. A61L 9/04; A61K 9/14; C07J 17/00

(52) U.S. Cl. 424/46; 540/114

(57) ABSTRACT

A compound of formula (I) 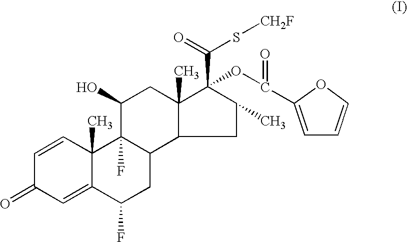 in the form of a substantially amorphous solid, and processes for the production thereof.

The present invention relates to a novel anti-inflammatory and anti-allergic compound of the androstane series and to processes for its preparation. The present invention also relates to pharmaceutical formulations containing the compound and to therapeutic uses thereof, particularly for the treatment of inflammatory and allergic conditions.

Glucocorticoids which have anti-inflammatory properties are known and are widely used for the treatment of inflammatory disorders or diseases such as asthma and rhinitis. For example, U.S. Pat. No. 4,335,121 discloses 6α, 9α-Difluoro-17α-(1-oxopropoxy)-11β-hydroxy-16α-methyl-3-oxo-androsta-1,4-diene-17β-carbothioic acid S-fluoromethyl ester (known by the generic name of fluticasone propionate) and derivatives thereof. The use of glucocorticoids generally, and especially in children, has been limited in some quarters by concerns over potential side effects. The side effects that are feared with glucocorticoids include suppression of the Hypothalamic-Pituitary-Adrenal (HPA) axis, effects on bone growth in children and on bone density in the elderly, ocular complications (cataract formation and glaucoma) and skin atrophy. Certain glucocorticoid compounds also have complex paths of metabolism wherein the production of active metabolites may make the pharmacodynamics and pharmacokinetics of such compounds difficult to understand. Whilst the modern steroids are very much safer than those originally introduced, it remains an object of research to produce new molecules which have excellent anti-inflammatory properties, with predictable pharmacokinetic and pharmacodynamic properties, with an attractive side effect profile, and with a convenient treatment regime.

We have now identified a novel glucocorticoid compound which substantially meets these objectives.

Thus, according to one aspect of the invention, there is provided a compound of formula (I) 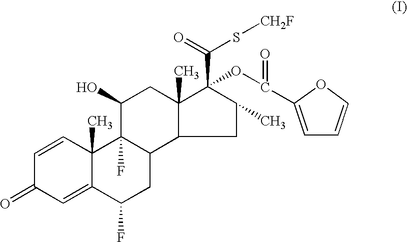
in the form of a substantially amorphous solid.

The chemical name of the compound of formula (I) is 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester.

The compound of formula (I) and formulations thereof have potentially beneficial anti-inflammatory or anti-allergic effects, particularly upon topical administration, demonstrated by, for example, its ability to bind to the glucocorticoid receptor and to illicit a response via that receptor, with long acting effect. Hence, the compound of formula (I) is useful in the treatment of inflammatory and/or allergic disorders, especially in once-per-day therapy.

The compound of formula (I) will preferably be present in the form of substantially amorphous solid particles. When intended for topical administration to the lung, the size of the amorphous solid particles will preferably be of controlled particle size suitable for this purpose. The optimum particle size for inhalation into the bronchial system is usually 1-20 μm, preferably 1-10 μm, especially 2-5 μm. Preferably the compound of formula (I) thereof will be present in the form of substantially amorphous solid particles having a mass median diameter (MMD) in the range 1-20 μm, more preferably 1-10 μm, especially 2-5 μm.

Most preferably the compound of formula (I) will be present in the form of substantially amorphous solid particles which are substantially spherical.

Substantially amorphous particles are particles containing a very low content of crystallinity, e.g. less than 20% crystallinity, preferably less than 10%, especially less than 5% e.g. less than 1% crystallinity. Crystallinity may be measured using methods familiar to those skilled in the art. These methods include, but are not limited to powder X-ray diffraction, differential scanning calorimetry, dynamic vapor sorption, isothermal microcalorimetry, inyerse gas chromatography, near infra-red spectroscopy and solid-state NMR.

Substantially spherical particles are defined by a radius measurement in each of the three orthogonal planes which is essentially the same e.g. the spread between the largest and smallest radius is less than 20% of the smallest radius, preferably less than 10%, especially less than 5%.

According to a further aspect of the invention there is provided a pharmaceutical formulation comprising a compound of formula (I) in the form of a substantially amorphous solid optionally together with one or more physiologically acceptable diluents or carriers.

The pharmaceutical formulation comprising a compound of formula (I) in the form of a substantially amorphous solid together with one or more physiologically acceptable diluents or carriers may be prepared by (i) mixing the compound of formula (I) in the form of a substantially amorphous solid with one or more solid physiologically acceptable diluents or carriers; or (ii) preparing a solid dispersion of compound of formula (I) in one or more diluents or carriers, for example, by spray drying a solution containing the compound of formula (I) and one or more physiologically acceptable diluents or carriers; or (iii) spray drying the compound of formula (I) in the form of a substantially amorphous solid suspended in a liquid having dissolved therein one or more physiologically acceptable diluents or carriers (so as to form a spray-coated product).

Thus as an aspect of the invention we provide (i) a pharmaceutical formulation comprising a compound of formula (I) in the form of a substantially amorphous solid together with one or more physiologically acceptable diluents or carriers obtainable by mixing the compound of formula (I) in the form of a substantially amorphous solid with one or more solid physiologically acceptable diluents or carriers; and (ii) a pharmaceutical formulation comprising a solid dispersion of compound of formula (I) in one or more diluents or carriers obtainable by spray drying a solution containing the compound of formula (I) and one or more physiologically acceptable diluents or carriers; and (iii) a pharmaceutical formulation comprising a compound of formula (I) in the form of a substantially amorphous solid together with one or more physiologically acceptable diluents or carriers obtainable by spray drying the compound of formula (I) in the form of a substantially amorphous solid suspended in a liquid having dissolved therein one or more physiologically acceptable diluents or carriers.

Example diluents or carriers include : polyethylene glycol, hydroxypropylmethyl cellulose, hydroxypropyl cellulose, methyl cellulose, polyvinylpyrrolidone, dibasic calcium phosphate, lactose, monosaccharide sugars eg mannitol, disaccharide sugars eg lactose, starch, amino acids and similar materials.

Pharmaceutical formulations for topical administration to the lung include dry powder compositions and spray compositions.

Dry powder compositions for topical delivery to the lung by inhalation may, for example, be presented in capsules and cartridges for use in an inhaler or insufflator of, for example, gelatine. Formulations generally contain a powder mix for inhalation of the compound of the invention and a suitable powder base (carrier substance) such as lactose or starch. Use of lactose is preferred. Each capsule or cartridge may generally contain between 20 μg-10 mg of the compound of formula (I) optionally in combination with another therapeutically active ingredient. Alternatively, the compound of the invention may be presented without excipients. Packaging of the formulation may be suitable for unit dose or multi-dose delivery. In the case of multi-dose delivery, the formulation can be pre-metered (e.g. as in Diskus, see GB 2242134 or Diskhaler, see GB 2178965, 2129691 and 2169265) or metered in use (e.g. as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

Pharmaceutical formulations which are non-pressurised and adapted to be administered as a dry powder topically to the lung via the buccal cavity (especially those which are free of excipient or are formulated with a diluent or carrier such as lactose or starch, most especially lactose) are of particular interest.

When an excipient such as lactose is employed, generally, the particle size of the exdipient will be much greater than the inhaled medicament within the present invention. When the excipient is lactose it will typically be present as milled lactose, wherein not more than 85% of lactose particles will have a MMD of 60-90 μm and not less than 15% will have a MMD of less than 15 μm.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, especially 1,1,1,2-tetrafluoroethane, 1,1,1,2,3,3,3-heptafluoro-n-propane or a mixture thereof. The aerosol composition may optionally contain additional formulation excipients well known in the art such as surfactants e.g. oleic acid or lecithin and cosolvents e.g. ethanol. One example formulation is excipient free and consists essentially of (e.g. consists of) compound of formula (I) (optionally together with a further active ingredient) and a propellant selected from 1,1,1,2-tetrafluoroethane, 1,1,1,2,3,3,3-heptafluoro-n-propane and mixture thereof. Another example formulation comprises particulate compound of formula (I), a propellant selected from 1,1,1,2-tetrafluoroethane, 1,1,1,2,3,3,3-heptafluoro-n-propane and mixture thereof and a suspending agent which is soluble in the propellant e.g. an oligolactic acid or derivative thereof as described in WO94/21229. The preferred propellant is 1,1,1,2-tetrafluoroethane. Pressurised formulations will generally be retained in a canister (e.g. an aluminium canister) closed with a valve (e.g. a metering valve) and fitted into an actuator provided with a mouthpiece.

The desirable biological properties of the compound of formula (I) are described below:

Compound (I) undergoes highly efficient hepatic metabolism to yield the 17-β carboxylic acid (X) as the sole major metabolite in rat and human in vitro systems. This metabolite has been synthesised and demonstrated to be >1000 fold less active than the parent compound in in vitro functional glucocorticoid assays.

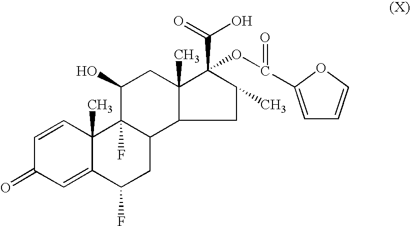

This efficient hepatic metabolism is reflected by in vivo data in the rat, which have demonstrated plasma clearance at a rate approaching hepatic blood flow and an oral bioavailability of <1%, consistent with extensive first-pass metabolism.

In vitro metabolism studies in human hepatocytes have demonstrated that compound (I) is metabolised in an identical manner to fluticasone propionate but that conversion of (I) to the inactive acid metabolite occurs approximately 5-fold more rapidly than with fluticasone propionate. This very efficient hepatic inactivation would be expected to minimise systemic exposure in man leading to an improved safety profile.

Inhaled steroids are also absorbed through the lung and this route of absorption makes a significant contribution to systemic exposure. Reduced lung absorption could therefore provide an improved safety profile. Studies with compound (I) have shown significantly lower exposure to compound (I) than with fluticasone propionate after dry powder delivery to the lungs of anaesthetised pigs.

Examples of disease states in which the compound of the invention has utility include inflammatory conditions of the nose, throat or lungs such as asthma (including allergen-induced asthmatic reactions), rhinitis (including hayfever), nasal polyps, chronic obstructive pulmonary disease (COPD), interstitial lung disease, and fibrosis.

The compound of formula (I) is expected to be most useful in the treatment of inflammatory disorders of the respiratory tract e.g. asthma and COPD, particularly asthma.

It will be appreciated by those skilled in the art that reference herein to treatment extends to prophylaxis as well as the treatment of established conditions.

As mentioned above, the compound of formula (I) is useful in human or veterinary medicine, in particular as an anti-inflammatory and anti-allergic agent.

There is thus provided as a further aspect of the invention the compound of formula (I) in the form of a substantially amorphous solid for use in human or veterinary medicine, particularly in the treatment of patients with inflammatory and/or allergic conditions.

According to another aspect of the invention, there is provided the use of the compound of formula (I) in the form of a substantially amorphous solid for the manufacture of a medicament for the treatment of patients with inflammatory and/or allergic conditions.

In a further or alternative aspect, there is provided a method for the treatment of a human or animal subject with an inflammatory and/or allergic condition, which method comprises administering to said human or animal subject an effective amount of the compound of formula (I) in the form of a substantially amorphous solid.

Further, there is provided a process for the preparation of such pharmaceutical compositions which comprises mixing the ingredients.

The proportion of the active compound of formula (I) in the local compositions according to the invention depends on the precise type of formulation to be prepared but will generally be within the range of from 0.001 to 10% by weight. Generally, however for most types of preparations advantageously the proportion used will be within the range of from 0.005 to 1% and preferably 0.01 to 0.5%. However, in powders for inhalation or insufflation the proportion used will usually be within the range of from 0.1 to 5%.

Aerosol formulations are preferably arranged so that each metered dose or “puff” of aerosol contains 1 μg-2000 μg e.g. 20 μg-2000 μg, preferably about 20 μg-500 μg of a compound of formula (I) optionally in combination with another therapeutically active ingredient. Administration may be once daily or several times daily, for example 2, 3, 4 or 8 times, giving for example 1, 2 or 3 doses each time. Preferably the compound of formula (I) is delivered once or twice daily. The overall daily dose with an aerosol will typically be within the range 10 μg-10 mg e.g. 100 μg-10 mg preferably, 200 μg-2000 μg.

Since the compound of formula (I) is long-acting, preferably the compound will be delivered once-per-day and the dose will be selected so that the compound has a therapeutic effect in the treatment of respiratory disorders (e.g. asthma or COPD, particularly asthma) over 24 hours or more.

The pharmaceutical compositions according to the invention may also be used in combination with another therapeutically active agent, for example, a β₂ adrenoreceptor agonist, an anti-histamine or an anti-allergic. The invention thus provides, in a further aspect, a combination comprising the compound of formula (I) together with another therapeutically active agent, for example, a β₂-adrenoreceptor agonist, an anti-histamine or an anti-allergic.

Examples of β₂-adrenoreceptor agonists include salmeterol (eg as racemate or a single enantiomer such as the R-enantiomer), salbutamol, formoterol, salmefamol, fenoterol or terbutaline and salts thereof, for example the xinafoate salt of salmeterol, the sulphate salt or free base of salbutamol or the fumarate salt of formoterol. Long-acting β₂-adrenoreceptor agonists are preferred, especially those having a therapeutic effect over a 24 hour period such as salmeterol or formoterol.

Preferred long acting β₂-adrenoreceptor agonists include those described in WO 02066422, WO02070490 and WO02076933.

Especially preferred long-acting β₂-adrenoreceptor agonists include compounds of formula(X): 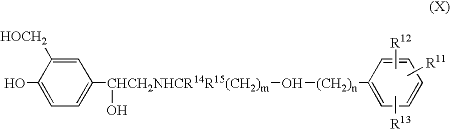
or a salt or solvate thereof, wherein:
m is an integer of from 2 to 8;
n is an integer of from 3 to 11,
with the proviso that m+n is 5 to 19,
R¹¹ is —XSO₂NR¹⁶R¹⁷ wherein X is —(CH₂)ₚ— or C₂₋₆ alkenylene;
R¹⁶ and R¹⁷ are independently selected from hydrogen, C₁₋₆alkyl, C₃₋₇cycloalkyl, C(O)NR¹⁸R¹⁹, phenyl, and phenyl (C₁₋₄alkyl)-,
or R¹⁶ and R¹⁷, together with the nitrogen to which they are bonded, form a 5-, 6-, or 7- membered nitrogen containing ring, and R¹⁶ and R17 are each optionally substituted by one or two groups selected from halo, C₁₋₆alkyl, C₁₋₆haloalkyl, C₁₋₆alkoxy, hydroxy-substituted C₁₋₆alkoxy, —CO₂R¹⁸, —SO₂NR¹⁸R¹⁹, —CONR¹⁸R¹⁹, —NR¹⁸C(O)R¹⁹, or a 5-, 6- or 7-membered heterocylic ring;
R18 and R¹⁹ are independently selected from hydrogen, C₁₋₆alkyl,
C₃₋₆cycloalkyl, phenyl, and phenyl (C₁₋₄alkyl)-; and
p is an integer of from 0 to 6, preferably from 0 to 4;
R¹² and R¹³ are independently selected from hydrogen, C₁₋₆-alkyl, C₁₋₆alkoxy, halo, phenyl, and C₁₋₆haloalkyl; and
R¹⁴ and R¹⁵ are independently selected from hydrogen and C₁₋₄alkyl with the proviso that the total number of carbon atoms in R¹⁴ and R¹⁵ is not more than 4.

Since the compound of formula (I) is long-acting, preferably the composition comprising the compound of formula (I) and the long-acting β₂-adrenoreceptor agonists will be delivered once-per-day and the dose of each will be selected so that the composition has a therapeutic effect in the treatment of respiratory disorders effect (e.g. in the treatment of asthma or COPD, particularly asthma) over 24 hours or more.

Examples of anti-histamines include methapyrilene or loratadine.

Other suitable combinations include, for example, other anti-inflammatory agents eg. NSAIDs (eg. PDE4 inhibitors, leukotriene antagonists, iNOS inhibitors, tryptase and elastase inhibitors, beta-2 integrin antagonists and adenosine 2a agonists)) or antiinfective agents (eg. antibiotics, antivirals).

Of particular interest is use of the compounds of formula (I) in combination with a phosphodiesterase 4 (PDE4) inhibitor. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family as well as PDE4. Generally it is preferred to use a PDE4 inhibitor which has an IC₅₀ ratio of about 0.1 or greater as regards the IC₅₀ for the PDE4 catalytic form which binds rolipram with a high affinity divided by the IC₅₀ for the form which binds rolipram with a low affinity. For the purposes of this disclosure, the cAMP catalytic site which binds R and S rolipram with a low affinity is denominated the “low affinity” binding site (LPDE 4) and the other form of this catalytic site which binds rolipram with a high affinity is denominated the “high affinity” binding site (HPDE 4). This term “HPDE4” should not be confused with the term “hPDE4” which is used to denote human PDE4.

Initial experiments were conducted to establish and validate a [³H]-rolipram binding assay. Details of this work are given in the Binding Assays described in detail below.

The preferred PDE4 inhibitors of use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE4 catalytic form which binds rolipram with a high affinity divided by the IC₅₀ for the form which binds rolipram with a low affinity.

A further refinement of this standard is that of one wherein the PDE4 inhibitor has an IC₅₀ ratio of about 0.1 or greater; said ratio is the ratio of the IC₅₀ value for competing with the binding of 1nM of [³H]R-rolipram to a form of PDE4 which binds rolipram with a high affinity over the IC₅₀ value for inhibiting the PDE4 catalytic activity of a form which binds rolipram with a low affinity using 1 μM[³H]-cAMP as the substrate.

Most preferred are those PDE4 inhibitors which have an IC₅₀ ratio of greater than 0.5, and particularly those compounds having a ratio of greater than 1.0. Preferred compounds are cis 4-cyano-4-(3-cyclopentyloxy4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]; these are examples of compounds which bind preferentially to the low affinity binding site and which have an IC₅₀ ratio of 0.1 or greater.

Other compounds of interest include:

Compounds set out in U.S. Pat. No. 5,552,438 issued 03 Sep. 1996; this patent and the compounds it discloses are incorporated herein in full by reference. The compound of particular interest, which is disclosed in U.S. Pat. No. 5,552,438, is cis-4-cyano-4-[3- (cyclopentyloxy)-4-methoxyphenyl]cyclohexane-1-carboxylic acid (also known as cilomalast) and its salts, esters, pro-drugs or physical forms;

AWD-12-281 from elbion (Hofgen, N. et al. 15th EFMC Int Symp Med Chem (Sep. 6-10, Edinburgh) 1998, Abst P. 98); a 9-benzyladenine derivative nominated NCS-613 (INSERM); D-4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (Sep. 19-23, Geneva) 1998] 1998, 12 (Suppl. 28): Abst P2393); roflumilast (CAS reference No 162401-32-3) and a pthalazinone (WO 9947505) from Byk-Gulden; or a compound identified as T-440 (Tanabe Seiyaku; Fuji, K. et al. J Pharmacol Exp Ther, 1998, 284(1): 162).

The combination referred to above may conveniently be presented for use in the form of a pharmaceutical formulation and thus pharmaceutical formulations comprising a combination as defined above together with a physiologically acceptable diluent or carrier represent a further aspect of the invention.

The compound according to the invention in combination with another therapeutically active ingredient as described above may be formulated for administration in any convenient way, and the invention therefore also includes within its scope pharmaceutical compositions comprising the compound of formula (I) in the form of a substantially amorphous solid in combination with another therapeutically active ingredient together, if desirable, in admixture with one or more physiologically acceptable diluents or carriers. The preferred route of administration for inflammatory disorders of the respiratory tract will generally be administration by inhalation.

Further, there is provided a process for the preparation of such pharmaceutical compositions which comprises mixing the ingredients.

Therapeutic agent combinations may be in any form, for example combinations may comprise a single dose containing separate particles of individual therapeutics, and optionally excipient material(s), alternatively, multiple therapeutics may be formed into individual multicomponent particles, formed for example by coprecipitation, and optionally containing excipient material(s).

The individual compounds of such combinations may be administered either sequentially in separate pharmaceutical compositions as well as simultaneously in combined pharmaceutical formulations. Appropriate doses of known therapeutic agents will be readily appreciated by those skilled in the art.

The compound of formula (I) in the form of a substantially amorphous solid be prepared by the methodology described hereinafter, constituting a further aspect of this invention.

The compound of formula (I) in the form of a substantially amorphous solid may be prepared by spray drying a solution containing the compound of formula (I). Any solvent that will dissolve the compound of formula (I) that can be evaporated safely in a spray drying process may be used. Suitable solvents for forming the solution include, but are not limited to, methyl acetate, ethyl acetate, isopropyl acetate, acetone, 2-butanone, 3-pentanone, 4-methyl-2-pentanone, ethanol, methanol, 1-propanol, isopropanol, acetonitrile, chloroform, dichloromethane especially methylethylketone (2-butanone). Solution concentration will typically be 0.5-15% more usually 0.5-10% especially 2-6% e.g. 3.5-4% w/w. The concentration that may be employed will be limited by the dissolution power of the solvent. Methylethylketone is preferred since it dissolves compound of formula (I) at a relatively high concentration which results in production advantages. Solubility of compound of formula (I) in the solvent may be enhanced by heating the solution. This may necessitate heating the appropriate parts of the apparatus (eg feed lines) to avoid unwanted precipitation of solids on cooling. The compound of formula (I) may be employed in non-solvated form or in the form of a solvate (e.g. with acetone). Preferably it is employed as the non-solvated Form 1 polymorph. Spray drying maybe performed, for example, using instruments supplied by Buchi or Niro. A pneumatic spray nozzle orifice of e.g. 0.04 inches (say 0.7-1.0 mm) is suitable, although alternate atomization methods such as rotary and pressure nozzles can be used. Solution flow rate. may typically be in the range 1-100 ml/min, especially 15-30 ml/min. The inlet temperature and flow rate combination should be suitable to evaporate the solvent completely to minimize the risk of solvent trapped in the particle expediting an amorphous to crystalline transition. Inlet temperatures can range from 50-250° C., typically 100-200° C.

As an aspect of the invention we also provide substantially amorphous particles of compound of formula (I) obtainable by performing an aforementioned process.

Compound of formula (I) may be prepared as follows:

A process for preparing a compound of formula (I) comprises alkylation of a thioacid of formula (II) 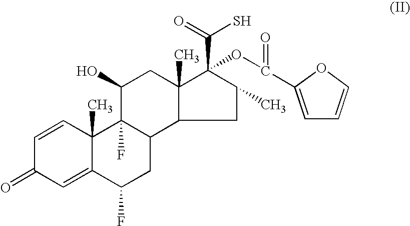
or a salt thereof.

In this process the compound of formula (II) may be reacted with a compound of formula FCH₂L wherein L represents a leaving group (e.g. a halogen atom, a mesyl or tosyl group or the like) for example, an appropriate fluoromethyl halide under standard conditions. Preferably, the fluoromethyl halide reagent is bromofluoromethane. Preferably the compound of formula (II) is employed as a salt, particularly the salt with diisopropylethylamine.

In a preferred process for preparing the compound of formula (I), the compound of formula (II) or a salt thereof is treated with bromofluoromethane optionally in the presence of a phase transfer catalyst. A preferred solvent is methylacetate, or more preferably ethylacetate, optionally in the presence of water. The presence of water improves solubility of both starting material and product and the use of a phase transfer catalyst results in an increased rate of reaction. Examples of phase transfer catalysts that may be employed include (but are not restricted to) tetrabutylammonium bromide, tetrabutylammonium chloride, benzyltributylammonium bromide, benzyltributylammonium chloride, benzyltriethylammonium bromide, methyltributylammonium chloride and methyltrioctylammonium chloride. THF has also successfully been employed as solvent for the reaction wherein the presence of a phase transfer catalyst again provides a significantly faster reaction rate. Preferably the product present in an organic phase is washed firstly with aqueous acid e.g. dilute HCl in order to remove amine compounds such as triethylamine and diisopropylethylamine and then with aqueous base e.g. sodium bicarbonate in order to remove any unreacted precursor compound of formula (II).

Compound of formula (I) in unsolvated form may be prepared by a process comprising:
(a) Crystallising the compound of formula (I) in the presence of a non-solvating solvent such as ethanol, methanol, water, ethyl acetate, toluene, methylisobutylketone or mixtures thereof; or
(b) Desolvating a compound of formula (I) in solvated form (e.g. in the form of a solvate with acetone, isopropanol, methylethylketone, DMF or tetrahydrofuran) e.g. by heating.

In step (b) the desolvation will generally be performed at a temperature exceeding 50° C. preferably at a temperature exceeding 100° C. Generally heating will be performed under vacuum.

Compound of formula (I) in unsolvated form has been found to exist in 3 crystalline polymorphic forms, Forms 1, 2 and 3, although Form 3 may be an unstable variant of Form 2. The Forms are characterised by their XRPD patterns shown in FIG. 4. Broadly speaking the Forms are characterised in their XRPD profiles as follows:
Form 1: Peak at around 18.9 degrees 2Theta
Form 2: Peaks at around 18.4 and 21.5 degrees 2Theta
Form 3: Peaks at around 18.6 and 19.2 degrees 2Theta.

Forms 1 appears to be the thermodynamically most stable form since Forms 2 and 3 are converted into Form 1 on heating.

A process for preparing a compound of formula (I) as crystalline unsolvated Form 1 polymorph comprises dissolving compound of formula (I) in methylisobutylketone, ethyl acetate or methyl acetate and producing compound of formula (I) as unsolvated Form 1 by addition of a non-solvating anti-solvent such as iso-octane or toluene.

According to a first preferred embodiment of this process the compound of formula (I) may be dissolved in ethyl acetate and compound of formula (I) as unsolvated Form 1 polymorph may be obtained by addition of toluene as anti-solvent. In order to improve the yield, preferably the ethyl acetate solution is hot and once the toluene has been added the mixture is distilled to reduce the content of ethyl acetate.

According to a second preferred embodiment of this process the compound of formula (I) may be dissolved in methylisobutylketone and compound of formula (I) as crystalline unsolvated Form 1 polymorph may be obtained by addition of isooctane as anti-solvent.

As well as the use of compound of formula (I) as a substantially amorphous solid in therapy, we have also appreciated a number of other advantageous uses.

As a further aspect of the invention we have also invented a new process for preparing compound of formula (I) as crystalline unsolvated form (typically Form 1 polymorph) from compound of formula (I) as a substantially amorphous solid. Thus the process comprises (i) heating the substantially amorphous solid, particularly at a temperature of between 90° C. and 160° C., until conversion is complete or (ii) contacting the substantially amorphous solid with vapours of a non-solvating solvent until conversion is complete. In step (i) if a temperature below 90° C. is used the conversion may not take place, or may take place incompletely. At above 160° C. chemical degradation may occur. The preferred temperature is between 90 and 100° C., especially around 95° C. The length of time necessary to achieve conversion will depend on the temperature, however will typically be 1-3 hours e.g. 2 hours at 95° C. Preferably the heating takes place in a controlled humidity enyironment. The time and temperature required to complete the amorphous to crystalline transition is dependent upon the process parameters used to produce the amorphous product and the resultant product thermal properties.

The temperature and time requirements for the conversion process can be decreased by the contacting the substantially amorphous solid with vapor of a non-solvating solvent (eg menthol, ethyl acetate, ethanol or methylisobutylketone (MIBK)).

Thus conversion to unsolvated polymorph can also be achieved without heating by contacting the compound of formula (I) as a substantially amorphous solid with vapours of a non-solvating solvent (eg menthol, ethyl acetate, ethanol or methylisobutylketone (MIBK) as per process (ii). The process normally results in generation of polymorph Form 1 (eg when ethyl acetate or MIBK are employed). However when ethanol is employed the process can result in generation of polymorph Form 2. Water is not suitable in this process; in fact compound of formula (I) in the form of a substantially amorphous solid appears to be quite stable in the presence of humidity. Process (ii) can normally be accelerated by heating eg up to around 70° C. For example when vapours of menthol are employed in this process the conversion to unsolvated polymorph Form 1 takes 24-48 hours at room temperature however is accelerated to less than 1 hour on heating to 50° C.

Agitation of the powder bed, by methods including, but not limited to vibrating, mixing or fluidization can be used in processes (i) and/or (ii) to minimize particle-particle contact and reduce the risk of bridging and subsequent particle size increase and loss of control during the surface crystallization process.

Appropriate temperature and rate of crystallization should be selected so as to maintaining size and surface control of the individual particles. The use of parameters that allow crystallisation to occur slowly will result in loss of spherical shape due to the production of fewer and larger individual crystals (see FIG. 7) therefore rapid crystallisation conditions are preferred (see FIG. 8). Overheating the particles should also be avoided since this can result in impurity formation.

A particularly preferred process for preparing compound of formula (I) as crystalline unsolvated Form 1 polymorph comprises:
(a) spray drying a solution containing compound of formula (I) so as to prepare compound of formula (I) as a substantially amorphous solid; and
(b) heating the substantially amorphous solid until conversion to compound of formula (I) as crystalline unsolvated Form 1 polymorph is completed.

Preferably in step (a) the compound of formula (I) in the form of substantially amorphous particles, most preferably particles which are of controlled particle size suitable for inhalation. Preferably the particles are substantially spherical.

Advantageously we have found that substantially amorphous particles of controlled particle size suitable for inhalation when subjected to process step (b) retain their size and shape and appear to be modified only in the respect of developing a roughened surface. Thus this process is especially suitable for preparing compound of formula (I) as crystalline unsolvated Form I polymorph in the form of particles of controlled particle size suitable for inhalation. This method of producing of particles of controlled particle size suitable for inhalation avoids the need to use wet processes employing special crystallisation conditions, or the need to use wasteful micronisation processes which may also result in loss of crystallinity in crystalline polymorph Form 1.

As an aspect of the invention we claim compound of formula (I) as particles of controlled particle size in the form of crystalline unsolvated Form 1 polymorph obtainable by such a process. We also provide pharmaceutical formulations containing such compound and particles. Such pharmaceutical formulations may be used in therapy for the treatment of inflammatory or allergic diseases such as those mentioned above.

Compound of formula (I) as a substantially amorphous solid for example particles of formula (I) as a substantially amorphous solid eg as formed by spray drying a solution containing a compound of formula (I) may also be used in the preparation of solutions containing compound of formula (I), especially solutions of compound of formula (I) in water. Such solutions may form the basis of pharmaceutical formulations which may also be used in therapy for the treatment of inflammatory or allergic diseases such as those mentioned above. We believe that compound of formula (I) in the form of a substantially amorphous solid, for example particles of formula (I) as a substantially amorphous solid eg as formed by spray drying a solution containing a compound of formula (I) may be dissolved more rapidly and/or dissolved to a greater extent in water with or without an agent to assist solubilisation relative to crystalline forms of compound of formula (I), particularly its unsolvated polymorph Form 1.

A process for preparing a compound of formula (I) as unsolvated Form 2 polymorph comprises dissolving compound of formula (I) in unsolvated form in methanol or dry dichloromethane and recrystallising the compound of formula (I) as unsolvated Form 2 polymorph. Typically the compound of formula (I) will be dissolved in hot methanol or dry dichloromethane and allowed to cool.

A process for preparing a compound of formula (I) as unsolvated Form 3 polymorph comprises dissolving compound of formula (I) in particular as the acetone solvate in dichloromethane in the presence of water (typically 1-3% water by volume) and recrystallising the compound of formula (I) as unsolvated Form 3 polymorph.

Compound of formula (I) in solvated form may be prepared by crystallising the compound of formula (I) from a solvating solvent such as acetone or tetrahydrofuran (THF).

Another process for preparing compound of formula (I) in solvated form (eg in the form of the solvate with acetone) comprises contacting the compound of formula (I) as a substantially amorphous solid with vapours of a solvating solvent (eg vapours of acetone).

Compounds of formula (II) may be prepared from the corresponding 17α-hydroxyl derivative of formula (III): 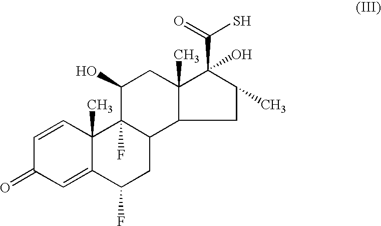
using for example, the methodology described by G. H. Phillipps et al., (1994) Journal of Medicinal Chemistry, 37, 3717-3729. For example the step typically comprises the addition of a reagent suitable for performing the esterification e.g. an activated derivative of 2-furoic acid such as an activated ester or preferably a 2-furoyl halide e.g. 2-furoyl chloride (employed in at least 2 times molar quantity relative to the compound of formula (III)) in the presence of an organic base e.g. triethylamine. The second mole of 2-furoyl chloride reacts with the thioacid moiety in the compound of formula (III) and needs to be removed e.g. by reaction with an amine such as diethylamine.

This method suffers disadvantages, however, in that the resultant compound of formula (II) is not readily purified of contamination with the by-product 2-furoyldiethylamide. We have therefore invented several improved processes for performing this conversion.

In a first such improved process we have discovered that by using a more polar amine such as diethanolamine, a more water soluble by-product is obtained (in this case 2-furoyldiethanolamide) which permits compound of formula (II) or a salt thereof to be produced in high purity since the by-product can efficiently be removed by water washing.

Thus we provide a process for preparing a compound of formula (II) which comprises:
(a) reacting a compound of formula (III) with an activated derivative of 2-furoic acid as in an amount of at least 2 moles of the activated derivative per mole of compound of formula (III) to yield a compound of formula (IIA) 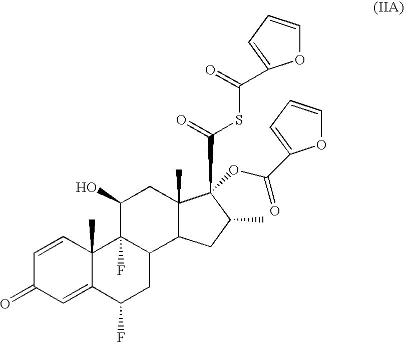
   ; and
(b) removal of the sulphur-linked 2-furoyl moiety from compound of formula (IIA) by reaction of the product of step (a) with an organic primary or secondary amine base capable of forming a water soluble 2-furoyl amide.

In two particularly convenient embodiments of this process we also provide methods for the efficient purification of the end product which comprise either
(c1) when the product of step (b) is dissolved in a substantially water immiscible organic solvent, purifying the compound of formula (II) by washing out the amide by-product from step (b) with an aqueous wash, or
(c2) when the product of step (b) is dissolved in a water miscible solvent, purifying the compound of formula (II) by treating the product of step (b) with an aqueous medium so as to precipitate out pure compound of formula (II) or a salt thereof.

In step (a) preferably the activated derivative of 2-furoic acid may be an activated ester of 2-furoic acid, but is more preferably a 2-furoyl halide, especially 2-furoyl chloride. A suitable solvent for this reaction is ethylacetate or methylacetate (preferably methylacetate) (when step (c1) may be followed) or acetone (when step (c2) may be followed). Normally an organic base e.g. triethylamine will be present. In step (b) preferably the organic base is diethanolamine. The base may suitably be dissolved in a solvent e.g. methanol. Generally steps (a) and (b) will be performed at reduced temperature e.g. between 0 and 5° C. In step (c1) the aqueous wash may be water, however the use of brine results in higher yields and is therefore preferred. In step (c2) the aqueous medium is for example a dilute aqueous acid such as dilute HCl.

We also provide an alternative process for preparing a compound of formula (II) which comprises:
(a) reacting a compound of formula (III) with an activated derivative of 2-furoic acid in an amount of at least 2 moles of activated derivative per mole of compound of formula (III) to yield a compound of formula (IIA); and
(b) removal of the sulphur-linked 2-furoyl moiety from compound of formula (IIA) by reaction of the product of step (a) with a further mole of compound of formula (III) to give two moles of compound of formula (II).

In step (a) preferably the activated derivative of 2-furoic acid may be an activated ester of 2-furoic acid, but is more preferably a 2-furoyl halide, especially 2-furoyl chloride. A suitable solvent for his step is acetone. Normally an organic base e.g. triethylamine will be present. In step (b) a suitable solvent is DMF or dimethylacetamide. Normally an organic base e.g. triethylamine will be present. Generally steps (a) and (b) will be performed at reduced temperature e.g. between 0 and 5° C. The product may be isolated by treatment with acid and washing with water.

This aforementioned process is very efficient in that it does not produce any furoylamide by-product (thus affording inter alia enyironmental advantages) since the excess mole of furoyl moiety is taken up by reaction with a further mole of compound of formula (II) to form an additional mole of compound of formula (II).

Further general conditions for the conversion of compound of formula (III) to compound of formula (II) in the two processes just described will be well known to persons skilled in the art.

According to a preferred set of conditions, however, we have found that the compound of formula (II) may advantageously be isolated in the form of a solid crystalline salt. The preferred salt is a salt formed with a base such as triethylamine, 2,4,6-trimethylpyridine, diisopropylethylamine or N-ethylpiperidine. Such salt forms of compound of formula (II) are more stable, more readily filtered and dried and can be isolated in higher purity than the free thioacid. The most preferred salt is the salt formed with diisopropylethylamine. The triethylamine salt is also of interest.

Compounds of formula (III) may be prepared in accordance with procedures described in GB 2088877B.

Compounds of formula (III) may also be prepared by a process comprising the following steps:

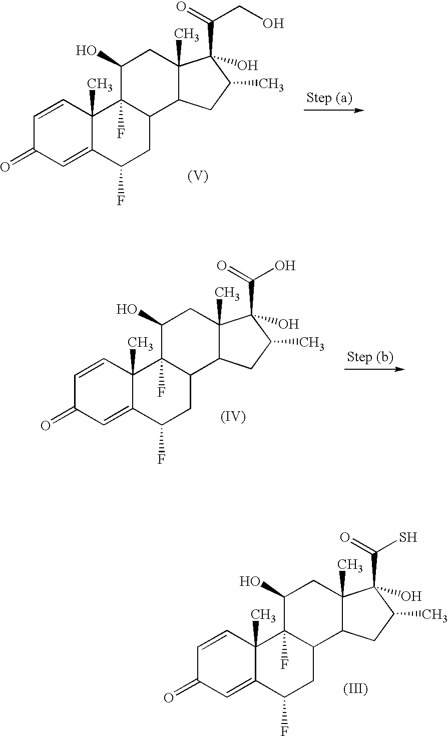

Step (a) comprises oxidation of a solution containing the compound of formula (V). Preferably, step (a) will be performed in the presence of a solvent comprising methanol, water, tetrahydrofuran, dioxan or diethylene glygol dimethylether. So as to enhance yield and throughput, preferred solvents are methanol, water or tetrahydrofuran, and more preferably are water or tetrahydrofuran, especially water and tetrahydrofuran as solvent. Dioxan and diethylene glygol dimethylether are also preferred solvents which may optionally (and preferably) be employed together with water. Preferably, the solvent will be present in an amount of between 3 and 10vol relative to the amount of the starting material (1wt.), more preferably between 4 and 6 vol., especially 5 vol. Preferably the oxidising agent is present in an amount of 1-9 molar equivalents relative to the amount of the starting material. For example, when a 50% w/w aqueous solution of periodic acid is employed, the oxidising agent may be present in an amount of between 1.1 and 10wt. relative to the amount of the starting material (1wt.), more preferably between 1.1 and 3wt., especially 1.3wt. Preferably, the oxidation step will comprise the use of a chemical oxidising agent. More preferably, the oxidising agent will be periodic acid or iodic acid or a salt thereof. Most preferably, the oxidising agent will be periodic acid or sodium periodate, especially periodic acid. Alternatively (or in addition), it will also be appreciated that the oxidation step may comprise any suitable oxidation reaction, e.g. one which utilises air and/or oxygen. When the oxidation reaction utilises air and/or oxygen, the solvent used in said reaction will preferably be methanol. Preferably, step (a) will inyolve incubating the reagents at room temperature or a little warmer, say around 25° C. e.g. for 2 hours. The compound of formula (IV) may be isolated by recrystallisation from the reaction mixture by addition of an anti-solvent. A suitable anti-solvent for compound of formula (IV) is water. Surprisingly we have discovered that it is highly desirable to control the conditions under which the compound of formula (IV) is precipitated by addition of anti-solvent e.g. water. When the recrystallisation is performed using chilled water (e.g. water/ice mixture at a temperature of 0-5° C.) although better anti-solvent properties may be expected we have found that the crystalline product produced is very voluminous, resembles a soft gel and is very difficult to filter. Without being limited by theory we believe that this low density product contains a large amount of solvated solvent within the crystal lattice. By contrast when conditions of around 10° C. or higher are used (e.g. around ambient temperature) a granular product of a sand like consistency which is very easily filtered is produced. Under these conditions, crystallisation typically commences after around 1 hour and is typically completed within a few hours (e.g. 2 hours). Without being limited by theory we believe that this granular product contains little or no solvated solvent within the crystal lattice.

Step (b) will typically comprise the addition of a reagent suitable for converting a carboxylic acid to a carbothioic acid e.g. using hydrogen sulphide gas together with a suitable coupling agent e.g. carbonyldiimidazole (CDI) in the presence of a suitable solvent e.g. dimethylformamide.

The advantages of the compound of formula (I) in the form of substantially amorphous solid may include the fact that the substance appears to demonstrate excellent anti-inflammatory properties, with predictable pharmacokinetic and pharmacodynamic behaviour, with an attractive side-effect profile, long duration of action, and is compatible with a convenient regime of treatment in human patients, in particular being amenable to once-per day dosing. Further advantages may include the fact that the substance has desirable physical and chemical properties which allow for ready manufacture and storage. In particular the amorphous solid is surprisingly resistant to conversion to crystalline form, and in particular is stable up to relatively high temperatures and over extended periods in the presence of a humid atmosphere. On the other hand conversion to a useful alternative crystalline form may be achieved under controlled conditions if desired thus rendering the compound of formula (I) in the form of substantially amorphous solid useful as a manufacturing intermediate in the preparation of other forms of compound of formula (I).

### BRIEF DESCRIPTION OF THE FIGURES

FIG. 1: Comparison of SEM images of compound of formula (I) crystalline acetone solvate, Form 1 (upper image) and amorphous material as obtained by spray drying (lower image)

FIG. 2: Comparison of XRPD profile of compound of formula (I) crystalline acetone solvate (upper trace) and amorphous material (lower trace)

FIG. 3: Temperature dependence of XRPD profile of compound of formula (I) amorphous material.

FIG. 4: Overlay of the XRPD profiles of Form 1, Form 2 and Form 3 polymorphs of unsolvated compound of formula (I)

FIG. 5: XRPD profile of amorphous material and material after 1 and 2 hours at 95° C. and a comparator trace of crystalline polymorph Form 1.

FIG. 6: SEM image of particles of compound of formula (I) as crystalline unsolvated Form 1 polymorph obtained by heating amorphous material (rapid crystal formation).

FIG. 7: SEM image of particles of compound of formula (I) as crystalline unsolvated Form 1 polymorph obtained by heating amorphous material showing example of large crystal growth from slow conversion process.

FIG. 8: SEM image of particles of compound of formula (I) as crystalline unsolvated Form 1 polymorph obtained by heating amorphous material. Example of small and rapid crystal formation and maintenance of spherical particle morphology.

FIG. 9: Particle size distribution of amorphous product.

The following non-limiting Examples illustrate the invention:

### EXAMPLES

### General

¹H-nmr spectra were recorded at 400 MHz and the chemical shifts are expressed in ppm relative to tetramethylsilane. The following abbreviations are used to describe the multiplicities of the signals: s (singlet), d (doublet), t (triplet), q (quartet), m (multiplet), dd (doublet of doublets), ddd (doublet of doublet of doublets), dt (doublet of triplets) and b (broad). Biotage refers to prepacked silica gel cartridges containing KP-Sil run on flash 12i chromatography module. LCMS was conducted on a Supelcosil LCABZ+PLUS column (3.3 cm ×4.6 mm ID) eluting with 0.1% HCO₂H and 0.01 M ammonium acetate in water (solvent A), and 0.05% HCO₂H 5% water in acetonitrile (solvent B), using the following elution gradient 0-0.7 min 0% B, 0.7-4.2 min 100% B, 4.2-5.3 min 0% B, 5.3-5.5 min 0% B at a flow rate of 3 ml/min. The mass spectra were recorded on a Fisons VG Platform spectrometer using electrospray positive and negative mode (ES+ve and ES−ve).

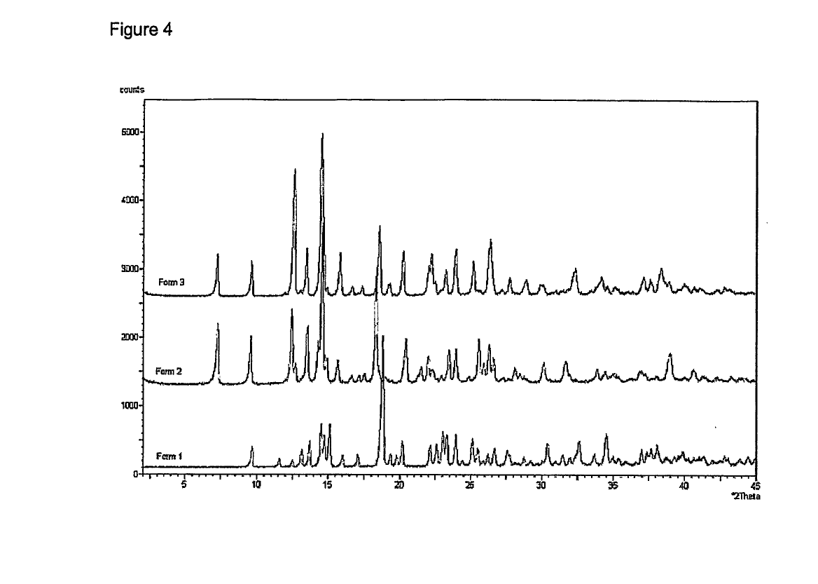

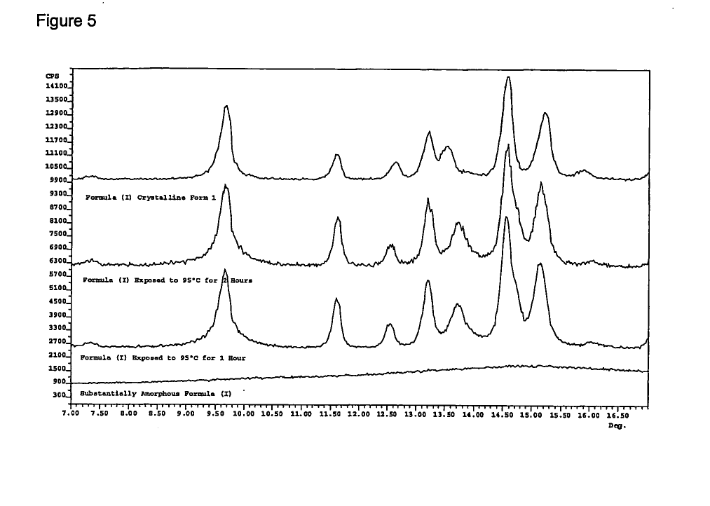

The XRPD analysis shown in the FIG. 4 was performed on a Phillips X'pert MPD powder diffractometer, serial number DY667. The method runs from 2 to 45 degrees 2Theta with 0.02 degree 2Theta step size and a 1 second collection time at each step. The XRPD analysis shown in FIG. 5 was performed on a Scintag PAD V powder diffractometer, serial number 40-6086. The X-ray source was a copper anode tube with a DGM-105 scintillation detector. The method was run from 2 to 50° 2-theta using continuous scan at 1° 2 theta/minute.

### Intermediates

### Intermediate 1: 6α, 9α-Difluoro-17α-[(2-furanylcarbonyl)oxy]-11β-hydroxy-16α-methyl-3-oxo-androsta-1,4-diene-17β-carbothioic acid diisopropylethylamine salt

A stirred suspension of 6α, 9α-difluoro-11β, 17α-dihydroxy-16α-methyl-3-oxo-androsta-1,4-diene-17β-carbothioic acid (prepared in accordance with the procedure described in GB 2088877B) (49.5 g) in methylacetate (500 ml) is treated with triethylamine (35 ml) maintaining a reaction temperature in the range 0-5° C. 2-Furoyl chloride (25 ml) is added and the mixture stirred at 0-5° C. for 1 hour. A solution of diethanolamine (52.8 g) in methanol (50 ml) is added and the mixture stirred at 0-5° C. for at least 2 hours. Dilute hydrochloric acid (approx 1M, 550 ml) is added maintaining a reaction temperature below 15° C. and the mixture stirred at 15° C. The organic phase is separated and the aqueous phase is back extracted with methyl acetate (2×250 ml). All of the organic phases are combined, washed sequentially with brine (5×250 ml) and treated with di-isopropylethylamine (30 ml). The reaction mixture is concentrated by distillation at atmospheric pressure to an approximate volume of 250 ml and cooled to 25-30° C. (crystallisation of the desired product normally occurs during distillation/subsequent cooling). Tertiary butyl methyl ether (TBME) (500 ml) is added, the slurry further cooled and aged at 0-5° C. for at least 10 minutes. The product is filtered off, washed with chilled TBME (2×200 ml) and dried under vacuum at approximately 40-50° C. (75.3 g, 98.7%). NMR (CDCl₃) δ: 7.54-7.46 (1H, m), 7.20-7.12 (1H, dd), 7.07-6.99 (1H, dd), 6.48-6.41 (2H, m), 6.41-6.32 (1H, dd), 5.51-5.28 (1H, dddd ²J_{H-F} 50 Hz), 4.45-4.33(1H, bd), 3.92-3.73 (3H, bm), 3.27-3.14 (2H, q), 2.64-2.12 (5H, m), 1.88-1.71 (2H, m), 1.58-1.15 (3H, s), 1.50-1.38 (15H, m), 1.32-1.23 (1H, m), 1.23-1.15 (3H s), 1.09-0.99 (3H, d)

### Intermediate 2: 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester

### Unsolvated Form 1

A mobile suspension of Intermediate 1 (12.61 g, 19.8 mmol) in ethyl acetate (230 ml) and water (50 ml) is treated with a phase transfer catalyst (benzyltributylammonium chloride, 10 mol %), cooled to 3° C. and treated with bromofluoromethane (1.10 ml, 19.5 mmol, 0.98 equivalents), washing in with prechilled (0° C.) ethyl acetate (EtOAc) (20 ml). The suspension is stirred overnight, allowing to warm to 17° C. The aqueous layer is separated and the organic phase is sequentially washed with 1M HCl (50 ml), 1% w/v NaHCO₃ solution (3×50 ml) and water (2×50 ml). The ethylacetate solution is distilled at atmospheric pressure until the distillate reaches a temperature of approximately 73° C. at which point toluene (150 ml) is added. Distillation is continued at atmospheric pressure until all remaining EtOAc has been removed (approximate distillate temperature 103° C.). The resultant suspension is cooled and aged at <10° C. and filtered off. The bed is washed with toluene (2×30 ml) and the product oven dried under vacuum at 60° C. to constant weight to yield the title compound (8.77 g, 82%) LCMS retention time 3.66 min, m/z 539 MH⁺, NMR δ (CDCl₃) includes 7.60 (1H, m), 7.18-7.11 (2H, m), 6.52 (1H, dd, J 4.2 Hz), 6.46 (1H, s), 6.41 (1H, dd, J 10, 2 Hz), 5.95 and 5.82 (2H dd, J 51, 9 Hz), 5.48 and 5.35 (1H, 2 m), 4.48 (1H, m), 3.48 (1H, m), 1.55 (3H, s), 1.16 (3H, s), 1.06 (3H, d, J 7 Hz).

### Intermediate 3: 6α, 9α-Difluoro-17α-[(2-furanylcarbonyl)oxy]-11β-hydroxy-16α-methyl-3-oxo-androsta-1,4-diene-17β-carbothioic acid

A stirred suspension of 6α, 9α-difluoro-11β, 17α-dihydroxy-16α-methyl-3-oxo-androsta-1,4-diene-17β-carbothioic acid (prepared in accordance with the procedure described in GB 2088877B) (1wt, 49.5 g) in acetone (10vol) is cooled to 0-5° C. and treated with triethylamine (0.51wt, 2.1eq), keeping the temperature below 5° C., and stirred for 5 min at 0-5°. 2-Furoyl chloride (0.65wt, 2.05eq) is then added over a minimum of 20 min, maintaining a reaction temperature at 0-5° C. The reaction mixture is stirred for at least 30 minutes and diluted with water (10vol) maintaining a reaction temperature in the range 0-5° C. The resultant precipitate is collected by filtration and washed sequentially with acetone/water (50/50 2vol) and water (2×2vol). The product is dried under vacuum at approximately 55° C. overnight to leave 6α, 9α-difluoro-17α-[(2-furanylcarbonyl)oxy]-11β-hydroxy-16α-methyl-3-oxo-androsta-1,4-diene-17β-yl S-(2-furanylcarbonyl) thioanhydride as a white solid (70.8 g, 98.2%) (NMR δ (CD₃CN) 0.99 (3H, d) (J=7.3 Hz), 1.24 (3H, s), 1.38 (1H, m) (J=3.9 Hz), 1.54 (3H, s), 1.67 (1H, m), 1.89 (1H, broad d) (J=15.2 Hz), 1.9-2.0 (1H, m), 2.29-2.45 (3H, m), 3.39 (1H, m), 4.33 (1H, m), 4.93 (1H, broad s), 5.53 (1H, ddd) ( J=6.9, 1.9 Hz; J_{HF}=50.9 Hz), 6.24 (1H, m), 6.29 (1H, dd) (J=10.3, 2.0 Hz), 6.63 (2H, m), 7.24-7.31 (3H, m), 7.79 (1H, dd) (J=<1 Hz), 7.86 (1H, dd) (J=<1 Hz)). A portion of the product (0.56 g) is mixed with 6α, 9α-difluoro-11β, 17α-dihydroxy-16α-methyl-3-oxo-androsta-1,4-diene-17β-carbothioic acid (0.41 g) in a 1:1 molar ratio in DMF (10 volumes wrt total steroid input). The reaction mixture is treated with triethylamine (approximately 2.1 equivalents) and the mixture is stirred at approximately 20° C. for approximately 6 hours. Water (50vol) containing excess conc HCl (0.5vol) is added to the reaction mixture and the resultant precipitate collected by filtration. The bed is washed with water (2×5vol) and dried in vacuo at approximately 55° C. overnight to leave the title compound as a white solid (0.99 g,102%).

### Intermediate 4: 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester

### Acetone Solvate

A solution of Intermediate 3 (530.1 g, 1wt) in dimethylformamide (DMF) (8vol) is treated with potassium hydrogen carbonate (0.202wt, 1.02eq) and the mixture cooled to −17±3° C. with stirring. Bromofluoromethane (BFM) (0.22wt, 0.99eq) is then added and the reaction stirred at −17±3° C. for at least 2 h. The reaction mixture is then added to water (17vol) at 5±3° C. over ca 10 min followed by a water (1vol) line wash. The suspension is stirred at 5-10° C. for at least 30 min and then filtered. The filter cake (the DMF solvate of 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester) is washed with water (4×4vol) and the product is pulled dry on the filter. The damp cake is returned to the vessel, acetone (5.75vol) added and heated at reflux for 2 h. The mixture is cooled to 52±3° C. and water (5.75vol) added, keeping temperature at 52±3° C. The mixture is then cooled to 20±3° C., filtered and dried in vacuo at 60±5° C. overnight to give the title compound as a white solid (556.5 g, 89%). NMR δ (CDCl₃) includes the peaks described in Intermediate 2 for the unsolvated compound and the following additional solvent peaks: 2.17 (6H, s).

### Pharmacological Activity

### In Vitro Pharmacological Activity

Pharmacological activity was assessed in a functional in vitro assay of glucocorticoid agonist activity which is generally predictive of anti-inflammatory or anti-allergic activity in vivo.

For the experiments in this section, compound of formula (I) was used as unsolvated Form 1 (Intermediate 2)

The functional assay was based on that described by K. P. Ray et al., Biochem J. (1997), 328, 707-715. A549 cells stably transfected with a reporter gene containing the NF-κB responsive elements from the ELAM gene promoter coupled to sPAP (secreted alkaline phosphatase) were treated with test compounds at appropriate doses for 1 hour at 37° C. The cells were then stimulated with tumour necrosis factor (TNF, 10ng/ml) for 16 hours, at which time the amount of alkaline phosphatase produced is measured by a standard colourimetric assay. Dose response curves were constructed from which EC₅₀ values were estimated.

In this test the compound of formula (1) showed an EC₅₀ value of <1nM.

The glucocorticoid receptor (GR) can function in at least two distinct mechanisms, by upregulating gene expression through the direct binding of GR to specific sequences in gene promoters, and by downregulating gene expression that is being driven by other transcription factors (such as NFκB or AP-1) through their direct interaction with GR.

In a variant of the above method, to monitor these functions, two reporter plasmids have been generated and introduced separately into A549 human lung epithelial cells by transfection. The first cell line contains the firefly luciferase reporter gene under the control of a synthetic promoter that specifically responds to activation of the transcription factor NFκB when stimulated with TNFα. The second cell line contains the renilla luciferase reporter gene under the control of a synthetic promotor that comprises 3 copies of the consensus glucocorticoid response element, and which responds to direct stimulation by glucocorticoids. Simultaneous measurement of transactivation and transrepression was conducted by mixing the two cell lines in a 1:1 ratio in 96 well plate (40,000 cells per well) and growing overnight at 37° C. Test compounds were dissolved in DMSO, and added to the cells at a final DMSO concentration of 0.7%. After incubation for 1 h 0.5ng/ml TNFα (R&D Systems) was added and after a further 15 hours at 37° C., the levels of firefly and renilla luciferase were measured using the Packard Firelite kit following the manufacturers' directions. Dose response curves were constructed from which EC₅₀ values were determined.

| | Transactivation | Transrepression |
|---|---|---|
| | (GR) | (NFκB) |
| | ED₅₀ (nM) | ED₅₀ (nM) |
| Compound of Formula (I) | 0.06 | 0.20 |
| Metabolite (X) | >250 | >1000 |
| Fluticasone propionate | 0.07 | 0.16 |

### In Vivo Pharmacological Activity

Pharmacological activity in vivo was assessed in an ovalbumin sensitised Brown Norway rat eosinophilia model. This model is designed to mimic allergen induced lung eosinophilia, a major component of lung inflammation in asthma.

For the experiments in this section, compound of formula (I) was used as unsolvated Form 1.

Compound of formula (I) produced dose dependant inhibition of lung eosinophilia in this model after dosing as an intra-tracheal (IT) suspension in saline 30 min prior to ovalbumin challenge. Significant inhibition is achieved after a single dose of 30 μg of compound of formula (I) and the response was significantly (p=0.016) greater than that seen with an equivalent dose of fluticasone propionate in the same study (69% inhibition with compound of formula (I) vs 41% inhibition with fluticasone propionate).

In a rat model of thymus involution 3 daily IT doses of 100 μg of compound (I) induced significantly smaller reductions in thymus weight (p=0.004) than an equivalent dose of fluticasone propionate in the same study (67% reduction of thymus weight with compound (I) vs 78% reduction with fluticasone propionate).

Taken together these results indicate a superior therapeutic index for compound (I) compared to fluticasone propionate.

### In vitro Metabolism In Rat And Human Hepatocytes

Incubation of compound (I) with rat or human hepatocytes shows the compound to be metabolised in an identical manner to fluticasone propionate with the 17-β carboxylic acid (X) being the only significant metabolite produced. Investigation of the rate of appearance of this metabolite on incubation of compound (I) with human hepatocytes (37° C., 10 μM drug concentration, hepatocytes from 3 subjects, 0.2 and 0.7 million cells/mL) shows compound (I) to be metabolised ca. 5-fold more rapidly than fluticasone propionate:—

| | | 17-β acid metabolite production | |
|---|---|---|---|
| Subject | Cell density | (pmol/h) | |
| number | (million cells/mL) | Compound (I) | Fluticasone propionate |
| 1 | 0.2 | 48.9 | 18.8 |
| 1 | 0.7 | 73.3 | 35.4 |
| 2 | 0.2 | 118 | 9.7 |
| 2 | 0.7 | 903 | 23.7 |
| 3 | 0.2 | 102 | 6.6 |
| 3 | 0.7 | 580 | 23.9 |

Median metabolite production 102-118 pmol/h for compound (I) and 18.8-23.0 pmol/h for fluticasone propionate.

### Pharmacokinetics After Intravenous (IV) And Oral Dosing In Rats

Compound (I) was dosed orally (0.1 mg/kg) and IV (0.1 mg/kg) to male Wistar Han rats and pharmacokinetic parameters determined. Compound (I) showed negligible oral bioavailability (0.9%) and plasma clearance of 47.3 mL/min/kg, approaching liver blood flow (plasma clearance of fluticasone propionate=45.2 mL/min/kg).

### Pharmacokinetics After Intra-Tracheal Dry Powder Dosing In the Pig

Anaesthetised pigs (2) were dosed intra-tracheally with a homogenous mixture of compound (I) (1 mg) and fluticasone propionate (1 mg) as a dry powder blend in lactose (10% w/w). Serial blood samples were taken for up to 8 h following dosing. Plasma levels of compound (I) and fluticasone propionate were determined following extraction and analysis using LC-MS/MS methodology, the lower limits of quantitation of the methods were 10 and 20pg/mL for compound (I) and fluticasone propionate respectively. Using these methods compound (I) was quantifiable up to 2 hours after dosing and fluticasone propionate was quantifiable up to 8 hours after dosing. Maximum plasma concentrations were observed for both compounds within 15 min after dosing. Plasma half-life data obtained from IV dosing (0.1 mg/kg) was used to calculate AUC (0-inf) values for compound (I). This compensates for the plasma profile of Compound (I) only being defined up to 2 hours after an IT dose and removes any bias due to limited data between compound (I) and fluticasone propionate.

Cₘₐₓ and AUC (0-inf) values show markedly reduced systemic exposure to compound (I) compared to fluticasone propionate:—

| | |
|---|---|
| Cmax | AUC (0-inf) |
| (pg/mL) | (hr · pg/mL) |

| | Pig 1 | Pig 2 | Pig 1 | Pig 2 |
|---|---|---|---|---|
| Compound of Formula (I) | 117 | 81 | 254 | 221 |
| Fluticasone propionate | 277 | 218 | 455 | 495 |

The pharmacokinetic parameters for both compound (I) and fluticasone propionate were the same in the anaesthetised pig following intravenous administration of a mixture of the two compounds at 0.1 mg/kg. The clearance of these two glucocorticoids is similar is this experimental pig model.

### EXAMPLES

### Example 1A

### 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester, amorphous particles

Intermediate 2 (30.04 g) was dissolved in methylethylketone (850 ml) to give a 3.5% solution. The solution was spray dried using a Niro Mobile Minor spray drier (Niro Inc, Columbia, Md., USA). The spray orifice was a two fluid pneumatic nozzle with 0.04 inch orifice diameter (Spraying Systems Inc, Wheaton, Ill., USA) . The other spray drying parameters were as follows:

Temperature: 150° C., outlet temperature 98° C.

Solution flow rate: 30 ml/min using Isco 260D syringe pump (Isco Inc, Lincoln, Nebr., USA)

Atomisation Pressure: 2 Bar

A white powder was recovered. System yield was 61%

Particle collection was achieved in the conventional manner using a Fisher Klosterman XQ120-1.375 high efficiency cyclone (Fisher-Klosterman Inc, Louisville, Ky., USA). The spray drying process was successful at producing smooth, spherical particles of amorphous 6α, 9α-Difluoro-17β-[(2-furanylcarbonyl)oxy]-11β-hydroxy-16α-methyl-3-oxo-androsta-1,4-diene-17β-carbothioic acid S-fluoromethyl ester.

### Example 1B

### 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester, amorphous particles

Intermediate 4 (1.26 g) was dissolved in methylethylketone (30 ml) to give a 3.8% solution. The solution was spray dried using a Buchi B-191 with spray nozle orifice diameter of 1.0 mm. The other spray drying parameters were as follows:

Temperature: 150° C., outlet temperature 106° C.

Solution flow rate: 15 ml/min

Atomisation Pressure: 2 Bar

Process gas flow rate 14 Cubic feet per minute (CFM)

A white powder was recovered from the cyclone and collection vessel, yield 37%.

### Example 1C

### 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester, amorphous particles

Intermediate 4 (10.03 g) was dissolved in methylethylketone (200mL) and stirred at room temperature. The resultant suspension was filtered resulting in a saturated solution. The solution was spray dried using a Buchi B-191 with spray nozzle orifice diameter of 0.7 mm. The other spray drying parameters were as follows:

Temperature: 200° C., outlet temperature 133° C.

Solution flow rate: 15 ml/min

Atomisation Pressure: 4 Bar

Process gas flow rate 20 Cubic feet per minute (CFM)

A white powder was recovered from the cyclone and collection vessel, yield 58%.

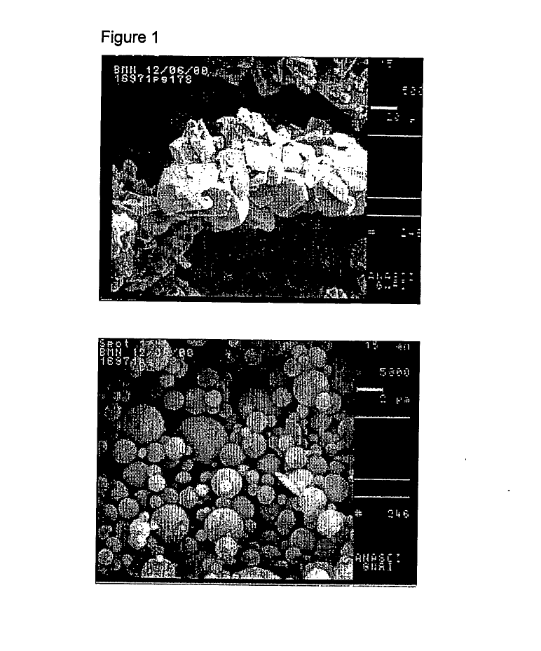

The starting material (Intermediate 4) and amorphous product (Example 1B) were studied by scanning electron microscopy using a Zeiss-Leo DSM 960 scanning electron microscope (SEM). Samples were prepared by placing approximately 50 mg onto carbon tape affixed to an aluminium stage. The samples were sputter coated with gold at 20 mA for 4 minutes. The samples were analysed in the SEM At 15 kV, 77 μA and 15 mm working distance. Images at 5000× magnification are shown in FIG. 1. The spray drying process was successful at producing smooth, spherical particles of amorphous 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester. The majority of the particles were between 0.5 and 4 μm.

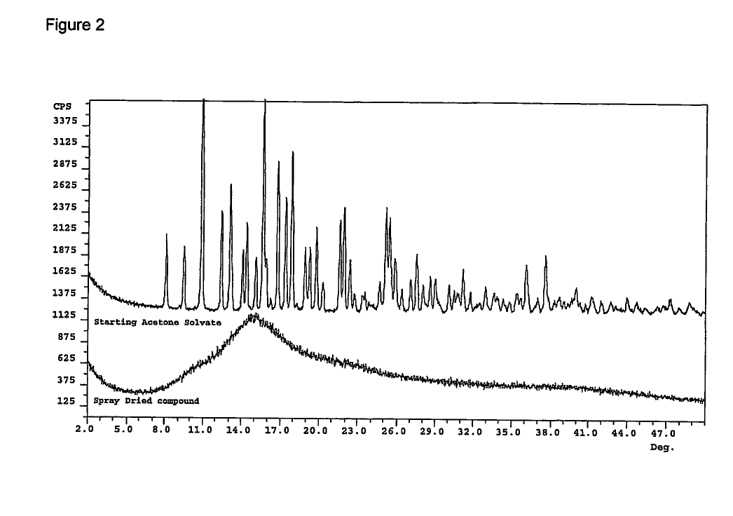

The starting material (Intermediate 4) and amorphous product (Example 1B) were studied by powder X-ray diffraction (XRD) using a Scintag XDS2000 diffractometer. The X-ray source was a copper anode tube with a DGM-105 scintillaction detector. Slit widths used were 1 mm, 2 mm, 0.5 mm and 0.3 mm for divergent incident, scatter incident, scatter diffracted and receiving respectively. The samples were prepared by lightly dusting a silicon wafer with the powder and lightly flattening the surface with a glass microscope slide. The wafer was fitted into a thermal control holder and the sample was scanned from 2 to 50 degrees 2 theta at 1 degree per minute. The XRD patterns are shown in FIG. 2. The top pattern represents the acetone solvate starting material and contains a high level of crystallinity. The spray dried powder, represented by the lower pattern has the halo characteristic of a highly disordered (amorphous) arrangement of molecules.

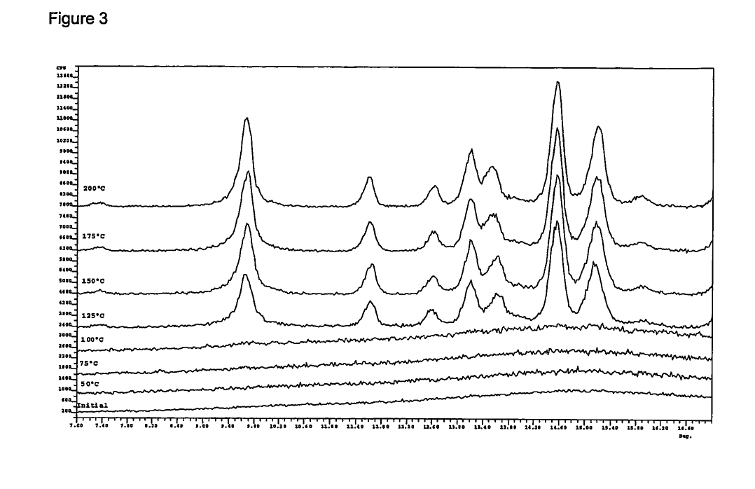

Amorphous product (Example 1B) was studied by hot stage X-ray powder diffraction to investigate the thermal stability of the product. The sample was heated to 50° C. and held for 5 minutes before analysis. The sample was analyzed from 7 to 17 °2 theta at 3 °2 theta per minute to minimize the changes that may occur from the beginning to the end of the run. The total run time was ˜9 minutes from heating to the end of the analysis. At the end of each run the temperature was increased 25° C. and the process was repeated up to 200° C. In the time frame allowed for each temperature, the sample converted to a crystalline form between 100 and 125° C. (See FIG. 3). At that point, the sample was cooled to room temperature and a complete 2 to 50° 2 theta scan was run to capture a larger d-spacing range with increased resolution. The powder pattern from the crystalline form does not match the starting acetone solvate and has been identified as non-solvate, Form 1.

Further studies have indicated that amorphous product is stable in a humid atmosphere. When amorphous product (Example 1B) was exposed to high humidity (humidity was step changed from 0 to 90% RH, 10% RH steps, 1 hr time hold at each step, cycle repeat twice to give total run time of around 42 hours) then relatively little water was taken up (around 1.6% w/w) and there was no change in appearance of product by SEM or enthalpy of crystallisation.

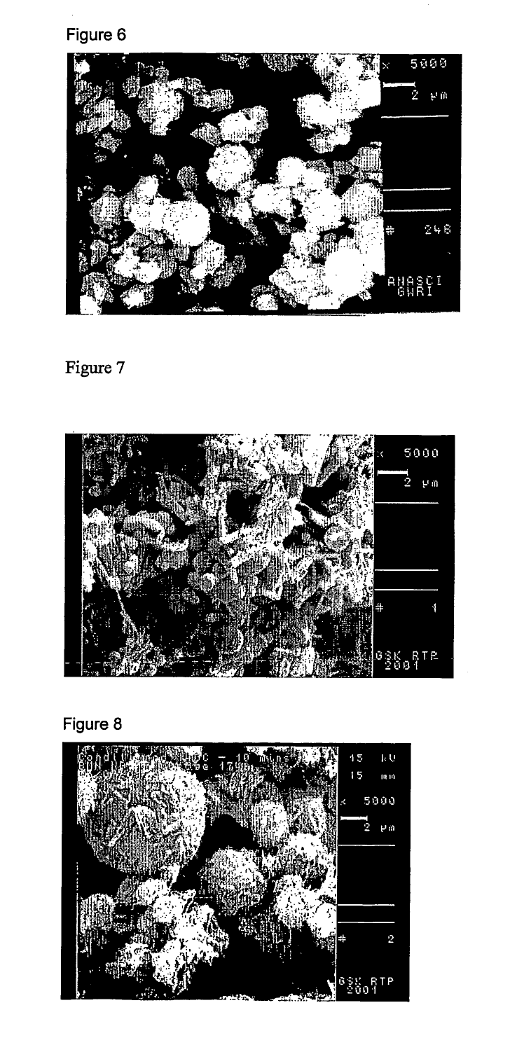

Amorphous product (Example 1B) was heated at 95° C. for up to 2 hours. The conversion to crystalline unsolvated Form 1 polymorph is demonstrated by the evolution of the XRPD pattern as shown in FIG. 5. The uppermost trace in FIG. 5 is of crystalline polymorph Form 1 for comparison purposes. The crystalline product which appears as spheres with roughened surfaces is shown in FIG. 6. The size and shape appears to be essentially unaltered relative to the amorphous starting product shown in FIG. 1.

FIGS. 7 and 8 provide examples of how the rate of conversion can effect the final particles. The powder in FIG. 7 was prepared by exposing the powder (Example 1C) to 70° C. for 24 hours. The resultant powder consists of smooth spherical particles that remain in the amorphous phase and large crystals in the shape of needles. Shape and potentially size control has been lost using this process. The powder in FIG. 8 was prepared by exposing the powder (Example 1C) to 140° C. for 10 minutes. It is believed that nucleation has occurred.

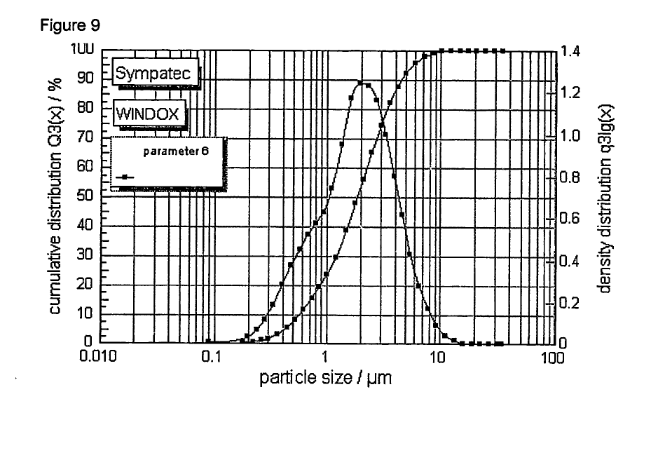

Particle size distribution of amorphous product (Example 1C) was studied using a laser diffraction particle sizing instrument (Sympatec (Princeton, N.J.)) with dry powder disperser (RODOS) using 3 mbar/100 mbar dispersion conditions. Results are shown in FIG. 9. The D₅₀ of this product was around 1.9 μm.

### Example 2

### Dry powder composition containing 6α, 9α-Difluoro- 17α-[(2-furanylcarbonyl)oxy]-11β-hydroxy-16α-methyl-3-oxo-androsta-1,4-diene-17β-carbothioic acid S-fluoromethyl ester, amorphous particles

A dry powder formulation may be prepared as follows:

| | |
|---|---|
| 6α,9α-Difluoro-17α-[(2-furanylcarbonyl)oxy]-11β- | 0.20 mg |
| hydroxy-16α-methyl-3-oxo-androsta-1,4-diene-17β- | |
| carbothioic acid S-fluoromethyl ester, amorphous particles | |
| prepared according to Example 1C: | |
| milled lactose (wherein not greater than 85% of particles | 12 mg |
| have a MMD of 60-90 μm, and not less than 15% of particles | |
| have a MMD of less than 15 μm): | |

A peelable blister strip containing 60 blisters each filled with a formulation as just described may be prepared.

### Example 3

### Dry powder composition containing 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester, amorphous particles and a long acting β₂-adrenoreceptor agonist

A dry powder formulation may be prepared as follows:

| | |
|---|---|
| 6α,9α-Difluoro-17α-[(2-furanylcarbonyl)oxy]-11β- | 0.20 mg |
| hydroxy-16α-methyl-3-oxo-androsta-1,4-diene-17β- | |
| carbothioic acid S-fluoromethyl ester, amorphous particles | |
| prepared according to Example 1C: | |
| Long-acting β₂-adrenoreceptor agonist | 0.02 mg |
| (micronised to a MMD of 3 μm): | |
| milled lactose (wherein not greater than 85% of particles | 12 mg |
| have a MMD of 60-90 μm, and not less than 15% of particles | |
| have a MMD of less than 15 μm): | |

A peelable blister strip containing 60 blisters each filled with a formulation as just described may be prepared.

### Example 4

### Aerosol formulation containing 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester, amorphous particles

An aluminium canister may be filled with a formulation as follows:

| | | |
|---|---|---|
| 6α,9α-Difluoro-17α-[(2-furanylcarbonyl)oxy]-11β- | 250 | μg |
| hydroxy-16α-methyl-3-oxo-androsta-1,4-diene-17β- | | |
| carbothioic acid S-fluoromethyl ester, amorphous | | |
| particles prepared according to Example 1C: | | |
| 1,1,1,2-tetrafluoroethane: | to 50 | μl |
| (amounts per actuation) | | |

in a total amount suitable for 120 actuations and the canister may be fitted with a metering valve adapted to dispense 50 μl per actuation.

### Example 5

### Aerosol formulation containing 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester, amorphous particles and a long acting β₂-adrenoreceptor agonist

An aluminium canister may be filled with a formulation as follows:

| | | |
|---|---|---|
| 6α,9α-Difluoro-17α-[(2-furanylcarbonyl)oxy]-11β- | 250 | μg |
| hydroxy-16α-methyl-3-oxo-androsta-1,4-diene-17β- | | |
| carbothioic acid S-fluoromethyl ester, amorphous particles | | |
| prepared according to Example 1C: | | |
| Long-acting β₂-adrenoreceptor agonist | 25 | μg |
| (micronised to a MMD of 3 μm): | | |
| 1,1,1,2-tetrafluoroethane: | to 50 | μl |
| (amounts per actuation) | | |

in a total amount suitable for 120 actuations and the canister may be fitted with a metering valve adapted to dispense 50 μl per actuation.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The patents and patent applications described in this application are herein incorporated by reference.

1. A compound of formula (I) 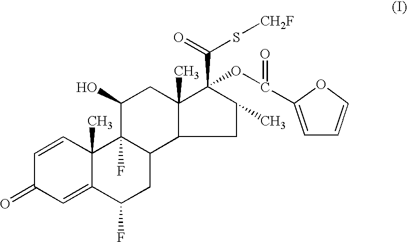
in the form of a substantially amorphous solid.

2. A compound of formula (I) as defined in claim 1 in the form of substantially amorphous solid particles.

3. A compound of formula (I) in the form of substantially amorphous solid particles according to claim 2 wherein the particles are of controlled particle size suitable for inhalation.

4. A compound of formula (I) in the form of substantially amorphous solid particles according to claim 2 which particles are substantially spherical.

5. A pharmaceutical formulation suitable for topical administration to the lung or nose comprising a compound of formula (I) according to claim 2 optionally together with one or more physiologically acceptable diluents or carriers.

6. A pharmaceutical formulation according to claim 5 in the form of a dry powder composition which contains a powder base.

7. A pharmaceutical formulation according to claim 6 wherein the powder base is lactose.

8. A pharmaceutical formulation according to claim 6 in the form of a pressurised aerosol formulation

9. A pharmaceutical formulation according to claim 8 which contains a liquefied fluorocarbon or hydrogen-containing chlorofluorocarbon propellant gas, or mixture thereof.

10. A process for preparing a compound of formula (I) in the form of a substantially amorphous solid, as claimed in claim 1, which comprises spray drying a solution containing compound of formula (I).

11. A process according to claim 10 wherein the solvent is methylethylketone.

12. A process according to claim 10 wherein the concentration of the solution is 2-4%.

13. Substantially amorphous particles of compound of formula (I) obtainable by performing a process as claimed in claim 12.

14. A method of treatment of respiratory disorders which comprises administering to a patient topically to the lung or nose a compound of formula (I) in the form of a substantially amorphous solid according to claim 1.

15. A process for preparing compound of formula (I) as a crystalline unsolvated form which comprises (i) heating compound of formula (I) in the form of a substantially amorphous solid until conversion to crystalline unsolvated Form 1 polymorph is complete; or (ii) contacting the compound of formula (I) as a substantially amorphous solid with vapours of a non-solvating solvent until conversion is complete.

16. A process for preparing compound of formula (I) as a crystalline unsolvated form which comprises:
(a) spray drying a solution containing compound of formula (I) so as to prepare compound of formula (I) as a substantially amorphous solid; and;
(b) (i) heating the substantially amorphous solid until conversion to compound of formula (I) as crystalline unsolvated form is completed; or (ii) contacting the compound of formula (I) as a substantially amorphous solid with vapours of a non-solvating solvent until conversion is complete.

17. A process according to claim 15 wherein step (b)(ii) takes place in the presence of heat.

18. A process according to claim 15 wherein in step (i) the compound is heated to a temperature of 90-160° C.

19. A process according to claim 15 wherein in step (a) the substantially amorphous solid is in the form of substantially amorphous particles which are of controlled particle size suitable for inhalation.

20. A process for preparing a formulation according to claim 5 which comprises (i) mixing the compound of formula (I) in the form of a substantially amorphous solid with one or more solid physiologically acceptable diluents or carriers; or (ii) preparing a solid dispersion of compound of formula (I) in one or more diluents or carriers by spray drying a solution containing the compound of formula (I) and one or more physiologically acceptable diluents or carriers; or (iii) spray drying the compound of formula (I) in the form of a substantially amorphous solid suspended in a liquid having dissolved therein one or more physiologically acceptable diluents or carriers.

21. A pharmaceutical formulation comprising a compound of formula (I) in the form of a substantially amorphous solid together with one or more physiologically acceptable diluents or carriers according to claim 5 obtainable (i) by mixing the compound of formula (I) in the form of a substantially amorphous solid with one or more solid physiologically acceptable diluents or carriers; or (ii) by spray drying a solution containing the compound of formula (I) and one or more physiologically acceptable diluents or carriers; or (iii) by spray drying the compound of formula (I) in the form of a substantially amorphous solid suspended in a liquid having dissolved therein one or more physiologically acceptable diluents or carriers.
